SEQUENCE LISTING (30 sequences)

SEQ ID NO: 1 - protein, 257 aa, Pyrococcus horikoshii
MKVERGDVIRLHYTGRVKETGQIFDTTYEEVAKEAGIYNPKGIYGPVPIIVGAGHVISGLDKRLVGLEVGKKYTLEVPPEEGFGLRDPKLIKVFTMGQFRKQGIVPFPGLEVEVTTDNGRKMKGRVITVSGGRVRVDFNHPLAGKTLIYEVEIVEKIEDPIEKIKALIELRLPMIDRDKVIIEVGEKDVKVNFGEQDVDPKTLILGEILLESDIKFLGYEKVEFKPSVEELLRPKQEEPVEEEKKEEQEESEEAQSS

SEQ ID NO: 2 - protein, 157 aa, Methanococcus jannaschii
LINLIKKGDYVKVDYILEVDGKVIDTSIEEVAKENKIYYPEREYEPIGFIVGNGELIEGFEEAVIGMEVGEEKTVTIPPEKGYGLRDERLIQEIPKEMFADADFEPQEGMLILASGIPAKIIKVTDDTVTLDFNHELAGKELKFTIKVRDVQPAESE

SEQ ID NO: 3 - protein, 432 aa, Escherichia coli
MQVSVETTQGLGRRVTITIAADSIETAVKSELVNVAKKVRIDGFRKGKVPMNIVAQRYGASVRQDVLGDLMSRNFIDAIIKEKINPAGAPTYVPGEYKLGEDFTYSVEFEVYPEVELQGLEAIEVEKPIVEVTDADVDGMLDTLRKQQATWKEKDGAVEAEDRVTIDFTGSVDGEEFEGGKASDFVLAMGQGRMIPGFEDGIKGHKAGEEFTIDVTFPEEYHAENLKGKAAKFAINLKKVEERELPELTAEFIKRFGVEDGSVEGLRAEVRKNMERELKSAIRNRVKSQAIEGLVKANDIDVPAALIDSEIDVLRRQAAQRFGGNEKQALELPRELFEEQAKRRVVVGLLLGEVIRTNELKADEERVKGLIEEMASAYEDPKEVIEFYSKNKELMDNMRNVALEEQAVEAVLAKAKVTEKETTFNELMNQQA

SEQ ID NO: 4 - DNA, 1299 bp, Escherichia coli
atgcaagtttcagttgaaaccactcaaggccttggccgccgtgtaacgattactatcgctgctgacagcatcgagaccgctgttaaaagcgagctggtcaacgttgcgaaaaaagtacgtattgacggcttccgcaaaggcaaagtgccaatgaatatcgttgctcagcgttatggcgcgtctgtacgccaggacgttctgggtgacctgatgagccgtaacttcattgacgccatcattaaagaaaaaatcaatccggctggcgcaccgacttatgttccgggcgaatacaagctgggtgaagacttcacttactctgtagagtttgaagtttatccggaagttgaactgcagggtctggaagcgatcgaagttgaaaaaccgatcgttgaagtgaccgacgctgacgttgacggcatgctggatactctgcgtaaacagcaggcgacctggaaagaaaaagacggcgctgttgaagcagaagaccgcgtaaccatcgacttcaccggttctgtagacggcgaagagttcgaaggcggtaaagcgtctgatttcgtactggcgatgggccagggtcgtatgatcccgggctttgaagacggtatcaaaggccacaaagctggcgaagagttcaccatcgacgtgaccttcccggaagaataccacgcagaaaacctgaaaggtaaagcagcgaaattcgctatcaacctgaagaaagttgaagagcgtgaactgccggaactgactgcagaattcatcaaacgtttcggcgttgaagatggttccgtagaaggtctgcgcgctgaagtgcgtaaaaacatggagcgcgagctgaagagcgccatccgtaaccgcgttaagtctcaggcgatcgaaggtctggtaaaagctaacgacatcgacgtaccggctgcgctgatcgacagcgaaatcgacgttctgcgtcgccaggctgcacagcgtttcggtggcaacgaaaaacaagctctggaactgccgcgcgaactgttcgaagaacaggctaaacgccgcgtagttgttggcctgctgctgggcgaagttatccgcaccaacgagctgaaagctgacgaagagcgcgtgaaaggcctgatcgaagagatggcttctgcgtacgaagatccgaaagaagttatcgagttctacagcaaaaacaaagaactgatggacaacatgcgcaatgttgctctggaagaacaggctgttgaagctgtactggcgaaagcgaaagtgactgaaaaagaaaccactttcaacgagctgatgaaccagcaggcgtaa

SEQ ID NO: 5 - protein, 270 aa, Escherichia coli
MKSLFKVTLLATTMAVALHAPITFAAEAAKPATAADSKAAFKNDDQKSAYALGASLGRYMENSLKEQEKLGIKLDKDQLIAGVQDAFADKSKLSDQEIEQTLQAFEARVKSSAQAKMEKDAADNEAKGKEYREKFAKEKGVKTSSTGLVYQVVEAGKGEAPKDSDTVVVNYKGTLIDGKEFDNSYTRGEPLSFRLDGVIPGWTEGLKNIKKGGKIKLVIPPELAYGKAGVPGIPPNSTLVFDVELLDVKPAPKADAKPEADAKAADSAKK

SEQ ID NO: 6 - DNA, 813 bp, Escherichia coli
atgaaatcactgtttaaagtaacgctgctggcgaccacaatggccgttgccctgcatgcaccaatcacttttgctgctgaagctgcaaaacctgctacaactgctgacagcaaagcagcgttcaaaaatgacgatcagaaatcagcttatgcactgggtgcttcgctgggtcgttacatggaaaactctcttaaagaacaagaaaaactgggcatcaaactggataaagatcagctgatcgctggtgttcaggatgcatttgctgataagagcaaactttccgaccaagagatcgaacagactctgcaagcattcgaagctcgcgtgaagtcttctgctcaggcgaagatggaaaaagacgcggctgataacgaagcaaaaggtaaagagtaccgcgagaaatttgccaaagagaaaggtgtgaaaacctcttccactggtctggtttatcaggtagtagaagccggtaaaggcgaagccccgaaagacagcgatactgttgtagtgaactacaaaggtacgctgatcgacggtaaagagttcgacaactcttacacccgtggtgaaccgctctctttccgtctggacggtgttatcccgggttggacagaaggtctgaagaacatcaagaaaggcggtaagatcaaactggttattccaccagaactggcttacggcaaagcgggtgttccggggatcccaccgaattctaccctggtgtttgacgtagagctgctggatgtgaaaccagcgccgaaggctgatgcaaagccggaagctgatgcgaaagccgcagactctgctaaaaaataa

SEQ ID NO: 7 - protein, 428 aa, Escherichia coli
MKNWKTLLLGIAMIANTSFAAPQVVDKVAAVVNNGVVLESDVDGLMQSVKLNAAQARQQLPDDATLRHQIMERLIMDQIILQMGQKMGVKISDEQLDQAIANIAKQNNMTLDQMRSRLAYDGLNYNTYRNQIRKEMIISEVRNNEVRRRITILPQEVESLAQQVGNQNDASTELNLSHILIPLPENPTSDQVNEAESQARAIVDQARNGADFGKLAIAHSADQQALNGGQMGWGRIQELPGIFAQALSTAKKGDIVGPIRSGVGFHILKVNDLRGESKNISVTEVHARHILLKPSPIMTDEQARVKLEQIAADIKSGKTTFAAAAKEFSQDPGSANQGGDLGWATPDIFDPAFRDALTRLNKGQMSAPVHSSFGWHLIELLDTRNVDKTDAAQKDRAYRMLMNRKFSEEAASWMQEQRASAYVKILSN

SEQ ID NO: 8 - DNA, 1287 bp, Escherichia coli
atgaagaactggaaaacgctgcttctcggtatcgccatgatcgcgaataccagtttcgctgccccccaggtagtcgataaagtcgcagccgtcgtcaataacggcgtcgtgctggaaagcgacgttgatggattaatgcagtcggtaaaactgaacgctgctcaggcaaggcagcaacttcctgatgacgcgacgctgcgccaccaaatcatggaacgtttgatcatggatcaaatcatcctgcagatggggcagaaaatgggagtgaaaatctccgatgagcagctggatcaggcgattgctaacatcgcgaaacagaacaacatgacgctggatcagatgcgcagccgtctggcttacgatggactgaactacaacacctatcgtaaccagatccgcaaagagatgattatctctgaagtgcgtaacaacgaggtgcgtcgtcgcatcaccatcctgccgcaggaagtcgaatccctggcgcagcaggtgggtaaccaaaacgacgccagcactgagctgaacctgagccacatcctgatcccgctgccggaaaacccgacctctgatcaggtgaacgaagcggaaagccaggcgcgcgccattgtcgatcaggcgcgtaacggcgctgatttcggtaagctggcgattgctcattctgccgaccagcaggcgctgaacggcggccagatgggctggggccgtattcaggagttgcccgggatcttcgcccaggcattaagcaccgcgaagaaaggcgacattgttggcccgattcgttccggcgttggcttccatattctgaaagttaacgacctgcgcggcgaaagcaaaaatatctcggtgaccgaagttcatgctcgccatattctgctgaaaccgtcgccgatcatgactgacgaacaggcccgtgtgaaactggaacagattgctgctgatatcaagagtggtaaaacgacttttgctgccgcagcgaaagagttctctcaggatccaggctctgctaaccagggcggcgatctcggctgggctacaccagatattttcgatccggccttccgtgacgccctgactcgcctgaacaaaggtcaaatgagtgcaccggttcactcttcattcggctggcatttaatcgaactgctggatacccgtaatgtcgataaaaccgacgctgcgcagaaagatcgtgcataccgcatgctgatgaaccgtaagttctcggaagaagcagcaagctggatgcaggaacaacgtgccagcgcctacgttaaaatcctgagcaactaa

SEQ ID NO: 9 - protein, 459 aa, Homo sapiens
MTAEEMKATESGAQSAPLPMEGVDISPKQDEGVLKVIKREGTGTEMPMIGDRVFVHYTGWLLDGTKFDSSLDRKDKFSFDLGKGEVIKAWDIAIATMKVGEVCHITCKPEYAYGSAGSPPKIPPNATLVFEVELFEFKGEDLTEEEDGGIIRRIQTRGEGYAKPNEGAIVEVALEGYYKDKLFDQRELRFEIGEGENLDLPYGLERAIQRMEKGEHSIVYLKPSYAFGSVGKEKFQIPPNAELKYELHLKSFEKAKESWEMNSEEKLEQSTIVKERGTVYFKEGKYKQALLQYKKIVSWLEYESSFSNEEAQKAQALRLASHLNLAMCHLKLQAFSAAIESCNKALELDSNNEKGLFRRGEAHLAVNDFELARADFQKVLQLYPNNKAAKTQLAVCQQRIRRQLAREKKLYANMFERLAEEENKAKAEASSGDHPTDTEMKEEQKSNTAGSQSQVETEA

SEQ ID NO: 10 - DNA, 1380 bp, Homo sapiens
atgacagccgaggagatgaaggcgaccgagagcggggcgcagtcggcgccgctgcccatggagggagtggacatcagccccaaacaggacgaaggcgtgctgaaggtcatcaagagagagggcacaggtacagagatgcccatgattggggaccgagtctttgtccactacactggctggctattagatggcacaaagtttgactccagtctggatcgcaaggacaaattctcctttgacctgggaaaaggggaggtcatcaaggcttgggacattgccatagccaccatgaaggtgggggaggtgtgccacatcacctgcaaaccagaatatgcctacggttcagcaggcagtcctccaaagattccccccaatgccacgcttgtatttgaggtggagttgtttgagtttaagggagaagatctgacggaagaggaagatggcggaatcattcgcagaatacagactcgcggtgaaggctatgctaagcccaatgagggtgctatcgtggaggttgcactggaagggtactacaaggacaagctctttgaccagcgggagctccgctttgagattggcgagggggagaacctggatctgccttatggtctggagagggccattcagcgcatggagaaaggagaacattccatcgtgtacctcaagcccagctatgcttttggcagtgttgggaaggaaaagttccaaatcccaccaaatgctgagctgaaatatgaattacacctcaagagttttgaaaaggccaaggagtcttgggagatgaattcagaagagaagctggaacagagcaccatagtgaaagagcggggcactgtgtacttcaaggaaggtaaatacaagcaagctttactacagtataagaagatcgtgtcttggctggaatatgagtctagtttttccaatgaggaagcacagaaagcacaggcccttcgactggcctctcacctcaacctggccatgtgtcatctgaaactacaggccttctctgctgccattgaaagctgtaacaaggccctagaactggacagcaacaacgagaagggcctcttccgccggggagaggcccacctggccgtgaatgactttgaactggcacgggctgatttccagaaggtcctgcagctctaccccaacaacaaagccgccaagacccagctggctgtgtgccagcagcggatccgaaggcagcttgcccgggagaagaagctctatgccaatatgtttgagaggctggctgaggaggagaacaaggccaaggcagaggcttcctcaggagaccatcccactgacacagagatgaaggaggagcagaagagcaacacggcagggagccagtctcaggtggagacagaagcatag

SEQ ID NO: 11 - protein, 370 aa, Homo sapiens
MSHPSPQAKPSNPSNPRVFFDVDIGGERVGRIVLELFADIVPKTAENFRALCTGEKGIGHTTGKPLHFKGCPFHRIIKKFMIQGGDFSNQNGTGGESIYGEKFEDENFHYKHDREGLLSMANAGRNTNGSQFFITTVPTPHLDGKHVVFGQVIKGIGVARILENVEVKGEKPAKLCVIAECGELKEGDDGGIFPKDGSGDSHPDFPEDADIDLKDVDKILLITEDLKNIGNTFFKSQNWEMAIKKYAEVLRYVDSSKAVIETADRAKLQPIALSCVLNIGACKLKMSNWQGAIDSCLEALELDPSNTKALYRRAQGWQGLKEYDQALADLKKAQGIAPEDKAIQAELLKVKQKIKAQKDKEKAVYAKMFA

SEQ ID NO: 12 - DNA, 1113 bp, Homo sapiens
atgtcgcacccgtccccccaagccaagccctccaaccccagtaaccctcgagtcttctttgacgtggacatcggaggggagcgagttggtcgaattgtcttagaattgtttgcagatatcgtacccaaaactgcggaaaattttcgtgcactgtgtacaggagaaaaaggcattggacacacgactgggaaacctctccatttcaaaggatgcccttttcatcgaattattaagaaatttatgattcagggtggagacttctcaaatcagaatgggacaggtggagaaagtatttatggtgaaaaatttgaagatgaaaatttccattacaagcatgatcgggagggtttactgagcatggcaaatgcaggccgcaacacaaacggttctcagttttttatcacaacagttccaactcctcatttggatgggaaacatgtggtgtttggccaagtaattaaaggaataggagtggcaaggatattggaaaatgtggaagtgaaaggtgaaaaacctgctaaattgtgcgttattgcagaatgtggagaattgaaggaaggagatgacgggggaatattcccaaaagatggctctggcgacagtcatccagatttccctgaggatgcggatatagatttaaaagatgtagataaaattttattaataacagaagacttaaaaaacattggaaatacttttttcaaatcccagaactgggagatggctattaaaaaatatgcagaagttttaagatacgtggacagttcaaaggctgttattgagacagcagatagagccaagctgcaacctatagctttaagctgtgtactgaatattggtgcttgtaaactgaagatgtcaaattggcagggagcaattgacagttgtttagaggctcttgaactagacccatcaaataccaaagcattgtaccgcagagctcaaggatggcaaggattaaaagaatatgatcaagcattggctgatcttaagaaagctcaggggatagcaccagaagataaagctatccaggcagaattgctgaaagtcaaacaaaagataaaggcacagaaagataaagagaaggcagtatatgcaaaaatgtttgcttag

SEQ ID NO: 13 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 14 - DNA, 1266 bp, Homo sapiens
atggatgtgctcagccctggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccaggtaacctgcgacctgttcatcgccctcgacgtgctgtgctgcacctcatccatcttgcacctgtgcgccatcgcgctggacaggtactgggccatcacggaccccatcgactacgtgaacaagaggacgccccggcgcgccgctgcgctcatctcgctcacttggcttattggcttcctcatctctatcccgcccatgctgggctggcgcaccccggaagaccgctcggaccccgacgcatgcaccattagcaaggatcatggctacactatctattccacctttggagctttctacatcccgctgctgctcatgctggttctctatgggcgcatattccgagctgcgcgcttccgcatccgcaagacggtcaaaaaggtggagaagaccggagcggacacccgccatggagcatctcccgccccgcagcccaagaagagtgtgaatggagagtcggggagcaggaactggaggctgggcgtggagagcaaggctgggggtgctctgtgcgccaatggcgcggtgaggcaaggtgacgatggcgccgccctggaggtgatcgaggtgcaccgagtgggcaactccaaagagcacttgcctctgcccagcgaggctggtcctaccccttgtgcccccgcctctttcgagaggaaaaatgagcgcaacgccgaggcgaagcgcaagatggccctggcccgagagaggaagacagtgaagacgctgggcatcatcatgggcaccttcatcctctgctggctgcccttcttcatcgtggctcttgttctgcccttctgcgagagcagctgccacatgcccaccctgttgggcgccataatcaattggctgggctactccaactctctgcttaaccccgtcatttacgcatacttcaacaaggactttcaaaacgcgtttaagaagatcattaagtgtaagttctgccgccag

SEQ ID NO: 15 - DNA, 40 bp, Artificial Sequence
  linker
ggactagtctggttccgcgtggatcccatatggaatccgg

SEQ ID NO: 16 - protein, 12 aa, Artificial Sequence
  translation of linker
TSLVPRGSHMEF

SEQ ID NO: 17 - DNA, 28 bp, Artificial Sequence
  PCR primer
ggccatgggaaaagttgaagctggtgat

SEQ ID NO: 18 - DNA, 26 bp, Artificial Sequence
  PCR primer
ccactagtagcttctgagtcctcttc

SEQ ID NO: 19 - DNA, 28 bp, Artificial Sequence
  PCR primer
ggccatgggccaagtttcagttgaaacc

SEQ ID NO: 20 - DNA, 28 bp, Artificial Sequence
  PCR primer
ccactagtcgcctgctggttcatcagct

SEQ ID NO: 21 - DNA, 27 bp, Artificial Sequence
  PCR primer
ggccatgggcacagccgaggagatgaa

SEQ ID NO: 22 - DNA, 27 bp, Artificial Sequence
  PCR primer
ccactagttgcttctgtctccacctga

SEQ ID NO: 23 - DNA, 24 bp, Artificial Sequence
  PCR primer
ggccatgggctcgcacccgtcccc

SEQ ID NO: 24 - DNA, 31 bp, Artificial Sequence
  PCR primer
ccactagtagcaaacatttttgcatatactg

SEQ ID NO: 25 - DNA, 32 bp, Artificial Sequence
  PCR primer
ggccatgggcaaatcactgtttaaagtaacgc

SEQ ID NO: 26 - DNA, 29 bp, Artificial Sequence
  PCr primer
ccactagtttttttagcagagtctgcggc

SEQ ID NO: 27 - DNA, 28 bp, Artificial Sequence
  PCR primer
ggccatgggcaagaactggaaaacgctg

SEQ ID NO: 28 - DNA, 29 bp, Artificial Sequence
  PCR primer
ccactagtgttgctcaggattttaacgta

SEQ ID NO: 29 - DNA, 26 bp, Artificial Sequence
  PCR primer
atcatatgaaatacctattgcctacg

SEQ ID NO: 30 - DNA, 33 bp, Artificial Sequence
  PCR primer
atgcggccgcctattactccagcttggtccctc

(19) United States
(12) Patent Application Publication
(10) Pub. No.: US 2005/0130259 A1
(43) Pub. Date: Jun. 16, 2005

(54) Expression vector, host, fused protein, process for producing fused protein and process for producing protein

(76) Inventors: Akira Ideno, Kamaishi-shi (JP); Tadashi Maruyama, Kamaishi-shi (JP); Masahiro Furutani, Mishima-gun (JP)

Correspondence Address: SUGHRUE MION, PLLC, 2100 PENNSYLVANIA AVENUE, N.W., SUITE 800, WASHINGTON, DC, 20037, US

(21) Appl. No.: 10/511,098

(22) PCT Filed: Jun. 25, 2003
(86) PCT No.: PCT/JP03/08020

(30) Foreign Application Priority Data
Jun. 25, 2002 (JP) 2002185020

(51) Int. Cl. C07H 21/04; C12P 21/04; C12P 21/06; C12N 9/16

(52) U.S. Cl. 435/68.1; 435/196; 435/69.7; 435/320.1; 536/23.2

(57) ABSTRACT

It is an object of the present invention to provide an expression vector, a host, a fused protein, a protein, a process for producing a fused protein, and a process for producing a protein, which can prevent formation of an unactive abnormal protein at production of a recombinant protein, and can produce a desired protein as a natural type, that is, a soluble type at a large amount and effectively. That is, the present invention is an expression vector, which comprises: (a) a first coding region encoding a polypeptide having molecular chaperone activity, and (b) a region having at least one restriction enzyme site in which a second coding region encoding a protein can be inserted. In the expression vector of the present invention, the first coding region is operatively linked to a promoter, and the restriction enzyme sites are in the same reading frame as the first coding region, and are downstream of the first coding region, or the restriction enzyme sites are disposed so that the inserted second coding region is operatively linked to a promoter, and the first coding region is in the same reading frame as the second coding region, and is downstream of the second coding region.

### TECHNICAL FIELD

The present invention relates to an expression vector, a host, a fused protein, a protein, a process for producing a fused protein and a process for producing a protein, which can prevent expression of a recombinant protein as an abnormal type of an inclusion body and the like, and can produce a recombinant protein as a natural type in a soluble fraction.

### BACKGROUND ART

Recently, genome analysis of various organisms has been completed, and it is considered that, from now on, study progresses towards covering functional analysis of a protein which is an expression product of a gene. Study in assisting analysis of life phenomenon by revealing property of individual proteins and, at the same time, analyzing interaction between proteins comprehensively has been rapidly increased. On the other hand, an intracellular receptor protein which specifically binds to various physiological active substances and transmits its action attracts an important interest in determining its three-dimensional structure because an active substance which binds to the receptor protein can be a candidate substance of a new medicament, and this protein is given an attention in screening of a novel medicament. When one tries to determine property of such the protein, such a method is general that a corresponding gene is inserted on a vector gene, transformed into a host such as bacterium, yeast and insect cell, and property of a recombinant protein obtained by expression is investigated.

Upon assessment of correct property of a protein, either the protein is folded into a correct steric structure or not becomes very important. However, when one tries to make a protein derived from a heterogenous organism by a protein expression method using the aforementioned host expression system, one often encounters the case where only an abnormal type protein having a different steric structure is obtained due to abnormal folding of a protein. It is known that such the protein is expressed as an aggregate called inclusion body in a host, and is degraded with a protease of a host cell. In order to solve them, it is considered that it is extremely important to control so that a reaction of folding a desired protein in a host cell is correctly performed.

When a desired protein is expressed as an inclusion body which is an abnormal type protein, as means for obtaining its normal type, a method of converting it into a normal type in vitro has been previously general. That is, this is a method of recovering an inclusion body from a host, solubilizing the inclusion body with guanidine hydrochloride or urea at a high concentration, and diluting with a suitable buffer to around 30 to 100-fold to re-fold a solubilized desired protein. As one example, an antibody is expected to be utilized in the medical field, but it is known that, when one tries to express its recombinant in a cytoplasm using Escherichia coli as a host, almost all of the recombinant is expressed as an insoluble inclusion body (Pluckthun, Biotechnology, 9, 545-, 1991). As a method of effectively re-folding an antibody obtained as an inclusion body in vitro, there is proposed a method of increasing a yield of a protein by containing chaperonin which promotes re-folding of the protein in a diluent (Japanese Kokai Publication Hei-9-220092). In addition to an antibody, various devices for increasing a yield of a desired protein by adding a reducing agent or an organic acid to a diluent have been proposed, such as a method of re-folding a NGF/BDNF family protein which is expressed as an inclusion body protein (Japanese Kokai Publication Hei-6-327489, Japanese Kokai Publication Hei-6-319549), and a method of re-folding neurotrophin-3 (Japanese Kokai Publication Hei-9-262093). However, these methods of re-folding a protein obtained as an inclusion body in vitro take a long time, but a resulting yield is low.

It has been reported that, in the case of an antibody, when an antibody is expressed in a periplasmic region by adding a signal sequence to its N-terminus, an antibody can be expressed in a soluble fraction even using Escherichia coli as a host (Glockshuber, Biochemistry 31, 1279-, 1992). However, since a periplasmic region is a very narrow region as compared with a cytoplasmic region, an amount of an expressed protein is very small and, even when an expression amount can be increased, a protein becomes an inclusion body. Some trials of expressing an antibody as a soluble entity in a cytoplasm have been reported. A device of increasing a yield of a soluble type by preventing of formation of an inclusion body from a recombinant antibody by coexpressing molecular chaperone which is involved in re-folding of a protein and an antibody gene in a cytoplasm, and a method of using a thioredoxin reductase-deficient strain as a host Escherichia coli have been proposed (Japanese Kokai Publication Hei-9-220092; Ploba, Gene 159, 203-, 1995). However, although these methods can afford a soluble type antibody, a yield of the antibody is very low as around 1 mg/l L medium (Levy, Protein Expression and Purification 23, 338-, 2001), and a method having a further better production efficacy is required.

On the other hand, since a membrane protein has nature of being present on the surface of a biomembrane or embedded in the interior of the biomembrane, and the protein has a high content of hydrophobic amino acids, it is known that the protein is often expressed as an inclusion body when expressed as a recombinant protein in the absence of a membrane. When toxicity to a cell is exhibited, it does not lead to expression in many cases. When a recombinant type of a membrane protein is obtained, the conventional means is to use a eukaryotic cell such as yeast cell and an animal cell and express the recombinant type in its membrane fraction. However, an expression amount is small, and cost and labor are necessary upon culturing expression and, therefore, a simpler expression method is demanded.

### SUMMARY OF THE INVENTION

In view of the aforementioned circumstances, an object of the present invention is to provide an expression vector, a host, a fused protein, a protein, a process for producing a fused protein, and a process for producing a protein, which can prevent formation of an unactive abnormal protein at production of a recombinant protein, and can produce a desired protein as a natural type, that is, a soluble type at a large amount and effectively.

That is, the present invention is an expression vector, which comprises (a) a first coding region encoding a polypeptide having molecular chaperone activity, and (b) a region having at least one restriction enzyme site in which a second coding region encoding a protein can be inserted.

In the expression vector of the present invention, the first coding region is operatively linked to a promoter, and the restriction enzyme site is in the same reading frame as the first coding region, and is downstream of the first coding region, or the restriction enzyme site is disposed such that the inserted second coding region is operatively linked to a promoter, and the first coding region is in the same reading frame as the second coding region, and is downstream of the second coding region.

The expression vector of the present invention preferably has a region being between the first coding region and the region having at least one restriction enzyme site in which the second coding region can be inserted, and being translated in the same reading frame to be a protease digestion site.

It is preferable that the express vector of the present invention has an inserted second coding region encoding a protein.

In the expression vector of the present invention, it is preferable that a polypeptide having a molecular chaperone activity is PPIase having molecular chaperone activity.

Examples of the PPIase having molecular chaperone activity include FKBP-type PPIase, cyclophilin-type PPIase, and parvulin-type PPIase.

Examples of the FKBP-type PPIase include archaebacterial FKBP-type PPIase, trigger factor-type PPIase, FkpA-type PPIase, and FKBP52-type PPIase.

Examples of the cyclophilin-type PPIase include Cyp40-type PPIase.

Examples of the parvulin-type PPIase include SurA-type PPIase.

Examples of the PPIase having chaperone activity also include PPIase comprising an IF domain and/or a C-terminal domain of archaebacterial FKBP-type PPIase, PPIase comprising a N-terminal domain and/or a C-terminal domain of trigger factor-type PPIase, PPIase comprising a N-terminal domain of FkpA-type PPIase, PPIase comprising a C-terminal domain of FKBP52-type PPIase, PPIase comprising a C-terminal domain of Cyp40-type PPIase, and PPIase comprising a N-terminal domain of SurA-type PPIase.

In the expression vector of the present invention, it is preferable that the second coding region has a nucleotide sequence encoding a monoclonal antibody, or a nucleotide sequence encoding a membrane protein.

A host containing the expression vector of the present invention is one of the present inventions.

It is preferable that the host of the present invention is Escherichia coli.

A fused protein comprising a polypeptide having the molecular chaperone activity and a protein encoded by a second coding region is also one of the present inventions.

It is preferable that the fused protein of the present invention comprises a protease digestion site.

A process for producing the fused protein of the present invention is one of the present inventions.

In the process for producing a fused protein of the present invention, it is preferable that a host containing the expression vector of the present invention is cultured under condition of expression of the expression vector, and the fused protein is expressed in a cytoplasm, or a region which is transcribed and translated to become a signal sequence is disposed at a 5′-terminus of a first coding region of the expression vector of the present invention or 5′-terminus of a second coding region, a host containing the resulting expression vector is cultured under condition of expression of the expression vector, and the fused protein is expressed in a periplasm or a medium, or the expression vector of the present invention is made to express the fused protein in a cell-free translation system.

In the process for producing a fused protein of the present invention, it is preferable that a fused protein is adsorbed onto a carrier harboring macrolide, cyclosporin, juglone or an analogous compound which inhibits PPIase activity, and then the carrier is recovered.

A process for producing a protein encoded by the second coding region, which comprises digesting a fused protein obtained by the process for producing a fused protein of the present invention with a protease which digests a protease digestion site, is also one of the present inventions.

### BRIEF DESCRIPTION OF THE DRAWING(S)

FIG. 1 is a view showing a gene arrangement of the vector TcFKfusion2 for making a fused protein with Thermococcus sp. KS-1 short type FKBP-type PPIase.

FIG. 2 is a view showing expression of TcFKBP18 in the case where TcFKfusion2 is used.

FIG. 3 is a view showing an electrophoretic pattern of a host protein.

FIG. 4 is a view showing expression of a fused protein of mouse anti-hen egg white lysozyme (HEL) Fab antibody fragment and TcFKBP18.

FIG. 5 is a view showing expression of mouse anti-hen egg white lysozyme (HEL) Fab antibody fragment alone.

FIG. 6 is a view showing expression of a fused protein of mouse anti-hen egg white lysozyme (HEL) scFv fragment and TcFKBP18.

FIG. 7 is a view showing a fused protein of purified mouse anti-HEL scFv and TcFKBP18, and results of treatment of the fused protein with thrombin.

FIG. 8 is a view showing activity of mouse anti-HEL scFv obtained as a result of expression, by an ELISA method.

### DETAILED DESCRIPTION OF THE INVENTION

The present invention will be described in detail below.

In the present invention, “operatively linked to a promoter” means that a first coding region is linked to a promoter so that a polypeptide having molecular chaperone activity is normally transcribed, or a second coding region is linked to a promoter so that a desired protein is normally transcribed.

In the present invention, “PPIase having molecular chaperone activity” includes those having substantially equivalent function. That is, substantially equivalent polypeptides, polypeptides comprising at least a part of them, and those in which a part of amino acids are altered to other amino acids, are included.

Further, in the present invention, a “domain” includes a domain having substantially equivalent function.

The expression vector of the present invention comprises (a) a first coding region encoding a polypeptide having molecular chaperone activity.

The molecular chaperone activity means activity of re-folding a denatured protein into an original natural type, or activity of inhibiting irreversible aggregation of a denatured protein. For example, rhodanese, citrate synthetase, malate dehydrogenase, glucose-6-phosphate dehydrogenase and the like are used as a model enzyme (Kawata, Bioscience and Industry 56, 593-, 1998), these are denaturation-treated with a protein denaturing agent such as 6M guanidine hydrochloride, thereafter, molecular chaperone activity of a test substrate can be assessed by a rate of regeneration of a denatured protein which is initiated upon dilution of a denaturing agent with a buffer containing the test substance, and a rate of inhibiting aggregation of a denatured protein. Examples of a method of assessing a rate of regeneration of a denatured protein include a method of Horowitz et al. (Horowitz, Methods Mol. Biol.40, 361-, 1995) in the case of rhodanese. Examples of a method of assessing inhibition of aggregation of a denatured protein include a method of Taguchi et al. (Taguchi, J. Biol. Chem.269, 8529-, 1994).

A polypeptide having the molecular chaperone activity is not particularly limited, and examples include PPIase having molecular chaperone activity such as archaebacterial FKBP-type PPIase; small heat shock protein, chaperonin, prefoldin, DnaK, DnaJ, GrpE, and HSP90.

It is reported that, in the small heat shock protein, around 24 to 32 of about 15 to 30 kDa subunits are assembled, and take a huge molecular structure, whereby, this has chaperone activity (Jakob, J. Biol. Chem. 268, 1517-, 1993). Crystallin having a region having high homology with this at its C-terminal region also has the same property as that of the small heat shock protein, and both of them can be applied to the expression vector of the present invention.

The chaperonin has a unique structure composed of a total 14 to 18 of subunits in which a double-toroidal structure of circularly linked 7 to 9 subunits is stacked. The chaperonin captures a denatured protein in a cavity of a toroidal structure, and promotes re-folding of a protein accompanied with consumption of a nucleotide such as ATP. In the case of eubacterium group 1-type chaperonin, it promotes a protein folding reaction further accompanied with binding of GroES (heat shock protein 10) as a cofactor. In the case of chaperonin, belonging to eukaryotic organism or archaebacterial group 2 type, it is known that the chaperonin effectively folds into a correct folded protein without a cofactor such as GroES (Gupta, Mol. Microbiol. 15, 1-, 1995).

The prefoldin is a molecular chaperone which was found to be a factor involved in the folding of a eukaryotic tubulin (Lopez, J. Struct. Biol. 135, 219-, 2001), and it is known that the prefoldin forms a hexamer, and has the chaperone activity to interact with a denatured protein in vitro (Siegert, Cell 103, 621-. 2000).

Homologues of the DnaK, DnaJ and GrpE are widely distributed in most of organisms, and are molecular chaperones which are thought to be involved in folding of proteins. Among them, in particular, the protein folding system of DnaK/DnaJ/GrpE system of Escherichia coli is well studied. As a proposed reaction mechanism for them, a nascent polypeptide biosynthesized in a ribosome binds to DnaK, DnaJ further binds thereto in the presence of ATP, whereby, irreversible aggregation formation of the peptide is inhibited. Further, accompanied with dissociation of a nucleotide depending on GrpE, a nascent polypeptide is also dissociated, and is transferred to a folding system of chaperonin (Fink, Molecular chaperones in the life cycle of proteins, MARCEL DEKKER, INC, 1998.) In the expression vector of the present invention, homologues which work like these Escherichia coli DnaK, DnaJ and GrpE can be used.

Some of the aforementioned HSP90 (heat shock protein 90) have the chaperone-like activity (Ramsey, J. Biol. Chem. 275, 17857-, 2000) and, in the expression vector of the present invention, homologues thereof, a part of them, or polypeptides containing them can be used.

PPIase (Peptidyl-prolyl cis-trans isomerase) having the molecular chaperone activity is one of protein folding factors involved in folding of a protein, and has activity of catalyzing a cis trans isomerization reaction of a N-terminal peptidyl bond of a proline residue (PPIase activity) among amino acids in a target protein during protein folding in a cell.

As a polypeptide having the molecular chaperone activity, inter alia, PPIase having molecular chaperone activity is preferable.

PPIase having the molecular chaperone activity is classified into three types, FK506 Binding Protein type (FKBP type), cyclophilin type and parvulin type based on sensitivity to it inhibitor. FKBP type PPIase is PPIase, activity of which is inhibited by FK506 which is one of immunological inhibitors, and homologues thereof. Cyclophilin type PPIase is PPIase having sensitivity to cyclosporine which is another immunological inhibitor, or homologues thereof. On the other hand, parvulin type PPIase is PPIase exhibiting no sensitivity to those immunological inhibitors, and activity of which is inhibited by juglone, or homologues thereof. These three kinds of PPIases have little homology on an amino acid primary sequence.

As PPIase having the molecular chaperone activity, among the aforementioned three kinds of PPIases, any type of PPIase may be used.

Examples of the FKBP-type PPIase include archaebacterial FKBP-type PPIase, trigger factor-type PPIase (Huang, Protein Sci. 9, 1254-, 2000), FkpA-type PPIase (Arie, Mol. Microbiol. 39, 199-, 2001), and FKBP52-type PPIase (Bose, Science 274, 1715-, 1996).

Examples of the cyclophilin-type PPIase include CyP40-type PPIase (Pirkl, J. Mol. Biol. 308, 795-, 2001).

Examples of the parvulin-type PPIase include SurA-type PPIase (Behrens, EMBO J. 20, 285-, 2001).

Regarding function of the archaebacterial FKBP-type PPIase, interestingly, it has been found that it has not only PPIase activity, but also molecular chaperone activity of inhibiting irreversible aggregation of a protein and, at the same time, promoting re-folding of a denatured protein (Furutani, Biochemistiry 39, 453-, 2000; Ideno, Eur. J. Biochem. 267, 3139-, 2000; Ideno, Biochem. J. 357, 465-,2001; Ideno, Appl. Env. Microbiol. 68, 464-, 2002). Molecular chaperone activity is originally activity which was found in protein folding system of chaperonin known one of molecular chaperone, and DnaK/DnaJ/GrpE system. These exerts function of supporting so that a polypeptide biosynthesized in a cell is folded into a correct form. Thereupon, hydrolysis of a high energy substance such as ATP is required. The archaebacterial FKBP-type PPIase is excellent in that a reaction of hydrolyzing the high energy substance is not required upon manifestation of the molecular chaperone activity.

The archaebacterial FKBP-type PPIase can be roughly classified into two kinds based on a difference in a molecular weight. One is a short type having a molecular weight of around 16 to 18kDa, and the other is a long type of around 26 to 33 kDa. As the archaebacterial FKBP-type PPIase used in the present invention, any of short type and long type FKBP-type PPIases may be used. However, generally, in view of two points that a short type tends to have stronger molecular chaperone activity, and as a molecular weight of a protein grows larger, an amount of expression of its recombinant protein tends to be decreased, the short type archaebacterial FKBP-type PPIase is preferable in the present invention. A width of the aforementioned molecular weight is a molecular weight width of the previously found PPIase, the archaebacterial FKBP-type PPIase in the present invention is not limited to this molecular weight width, any may be used as long as it belongs to substantially the same group.

The archaebacterial FKBP-type PPIase is not particularly limited, but may be derived from any archaebacteria. For example, among the previously found archaebacterial FKBP-type PPIases, examples of a short type include PPIases derived from thermophilic and hyperthermophilic archaebacteria such as Methanococcus thermolithotrophicus, Thermococcus sp. KS-1, and Methanococcus jannaschii, and derived from mesophilic archaebacteria such as Methanosarcina mazei, Methanosarcina acetivorans, and Methanosarcina barkeri (Maruyama, Front. Biosci 5, 821-, 2000). On the other hand, as a result of genome analysis and other analysis, a long type has been found on a genome of almost all archaebacteria. Examples include PPIases derived from thermophilic and hyperthermophilic archaebacteria such as Pyrococcus horikoshii, Aeropyrum pernix, Sulfolobus solfataricus, Methanococcus jannaschii, Archaeoglobus fulgidus, Methanobacterium autotrophicum, Thermoplasma acidophilum, and derived from mesophilic archaebacteria such as Halobacterium cutirubrum (Maruyama, Front. Biosci 5, 821-,2000). Inter alia, PPIase derived from mesophilic archaebacteria is preferable. As one example of the aforementioned long type archaebacterial FKBP-type PPIase, an amino acid sequence derived from Pyrococcus horikoshii is shown in SEQ ID NO: 1 and, as one example of the aforementioned short type archaebacterial FKBP-type PPIase, an amino acid sequence derived from Methanococcus jannaschaii is shown in SEQ ID NO: 2.

The archaebacterial FKBP-type PPIase has a FKBP domain involved in PPIase activity and binding with FK506, and an IF domain, and the long type archaebacterial FKBP-type PPIase has further a C-terminal domain (Maruyama and Furutani, Front Biosci. 1, D821-, 2000). The IF domain (Insert in the flap; Suzuki, J. Mol. Biol.328, 1149-, 2003) comprises about 100 amino acids inserted in an amino acid sequence constituting a FKBP domain, on an amino acid primary sequence, and forms a characteristic high order structure forming a domain structure (Maruyama and Furutani, Front Biosci. 1, D821-, 2000; Insert in the flap; Suzuki, J. Mol. Biol. 328, 1149-, 2003). It has been revealed that a FK506 binding region of a FKBP domain and an IF domain are involved in molecular chaperone activity of the short type archaebacterial FKBP-type PPIase (Furutani, Biochemistry 39, 2822-, 2000; Ideno, Biochem. J. 357, 465-, 2001). In addition, it has been revealed that, regarding the long type archaebacterial FKBP-type PPIase, a C-terminal domain together with the aforementioned two domains is involved in the molecular chaperone activity (IDENO, Eur. J. Biochem. 267, 3139-, 2000). In the present invention, PPIase comprising an IF domain and/or a C-terminal domain of archaebacterial FKBP-type PPIase can be used as PPIase having the molecular chaperone activity. For example, chimera PPIase in which an IF domain and a C-terminal domain are introduced by protein engineering into human FKBP12 which is PPIase originally having no molecular chaperone activity can be used as PPIase having the molecular chaperone activity. As an IF domain of the archaebacterial FKBP-type PPIase, a region of 78^{th} proline to 140^{th} tyrosine, in SEQ ID NO: 1, and a region of 78^{th} proline to 141^{st} glutamic acid in SEQ ID NO: 2 corresponds to IF domain respectively (IDENO, Eur. J. Biochem. 267, 3139-, 2000). On the other hand, as a C-terminal domain of the archaebacterial FKBP-type PPIase, a region of 157^{th} isoleucine to a C-terminus in SEQ ID NO: 1 corresponds to a C-terminal domain (IDENO, Eur. J. Biochem. 267, 3139-,2000). Homology of each domain can be determined by using multiple alignment software such as ClustalW.

The trigger factor-type PPIase is PPIase which has been found on a genome of almost all bacteria. The trigger factor-type PPIase is not particularly limited, and examples include PPIases derived from Escherichia coli, derived from Mycoplasma genitalium, derived from Bacillus subtilis, derived from Salmonella enterica, derived from Staphylococcus aureus, derived from Mycobacterium leprae, derived from Agrobacterium tumefacium, derived from Lactococcus lactis, derived from Campyrobacter jejuni, derived from Streptococcus pyogenes, and derived from Corynebacterium diphtheriae. In addition, as the trigger factor-type PPIase used in the present invention, any trigger factor-type PPIase may be used as long as it belongs to a group which is recognized to be substantially as same as that of bacterial trigger factor in an amino acid sequence. As one examples of the trigger factor-type PPIase, an amino acid sequence of Escherichia coli trigger factor type PPIase is shown in SEQ ID NO: 3, and the nucleotide sequence is shown in SEQ ID NO: 4.

The trigger factor-type PPIase has a FKBP domain involved in PPIase activity and binding with FK506 as a middle domain, and two domains on its N-terminal sides and C-terminal side, respectively (Zarnt, J. Mol. Biol.271, 827-, 1997). It is known that molecular chaperone activity of the trigger factor-type PPIase is activity independent of PPIase activity like archaebacterial FKPB-type PPIase, and it is suggested that the molecular chaperone activity is due to action of any one or both of its N-terminal domain and it C-terminal domain. In the present invention, PPIase comprising a N-terminal domain and/or a C-terminal domain of trigger factor-type PPIase can be used as PPIase having the molecular chaperone activity. As a N-terminal domain and a C-terminal domain of the trigger factor-type PPIase, in SEQ ID NO: 3, a region of first methionine to around 145^{th} arginine corresponds to a N-terminal domain, and a region of around 252^{nd} phenylalanine to a C-terminus corresponds to a C-terminal domain (Zarnt, J. Mol. Biol.271, 827-, 1997). Homology of each domain can be determined by using multiple alignment software such as ClustalW.

The aforementioned FkpA-type PPIase and SurA-type PPIase are both PPIase expressed in a periplasmic region of Gram-negative bacteria including Escherichia coli. The FkpA-type PPIase is FKBP-type PPIase, activity of which is inhibited by FK506, while SurA-type PPIase is one of parvulin-type PPIase homologues exhibiting no sensitivity to an immunological inhibitor of any of FK506 and cyclosporine which is other immunological inhibitor. These two PPIases are also known as a protein exhibiting molecular chaperone activity (Ramm, J. Biol. Chem. 275, 17106-, 2000; Behrens, EMBO. J.20, 285-, 2001).

The FkpA-type PPIase and SurA-type PPIase are not only found in a genome of Gram-negative bacteria, but also homologues thereof are found in a genome of a eukaryote such as yeast.

FkpA-type PPIase and SurA-type PPIase used in the present invention are not particularly limited, and examples include PPIases derived from Escherichia coli, derived from Pyrobaculum aerophilium, derived from Pseudomonas aeruginosa, derived from Xylella fastidiosa, derived from Neisseria meningitides, derived from Mesorhizobium loti, derived from Heamophilus influenzae, and derived from Ralstonia solanacearum. In addition, not only PPIase derived from bacteria, but also PPIase derived from any organism may be used as long as it belong to the same group as that of them, and has substantially the same function. As one example of the FkpA-type PPIase, an amino acid sequence derived from Escherichia coli is shown in SEQ ID NO: 5, and the nucleotide sequence is shown in SEQ ID NO: 6. In addition, as one example of the SurA-type PPIase, an amino acid sequence derived from Escherichia coli is shown in SEQ ID NO: 7 and the nucleotide sequence is shown in SEQ ID NO: 8.

The FkpA-type PPIase has a FKBP domain on its C-terminal side and a N-terminal domain other than the domain (Arie, Mol. Microbiol. 39, 199-, 2001). It is known that molecular chaperone activity of the FkpA-type PPIase is also activity independent of PPIase activity, and it is suggested that its N-terminal domain is involved in the molecular chaperone activity. In the present invention, PPIase comprising a N-terminal domain of FkpA-type PPIase can be used as PPIase having the molecular chaperone activity. As a N-terminal domain of the FkpA-type PPIase, in SEQ ID NO: 5, a region of a N-terminus to around 120^{th} aspartic acid corresponds to a N-terminal domain (Arie, Mol. Microbiol. 39, 199-, 2001).

On the other hand, the SurA-type PPIase also has a domain having high homology with parvulin-type PPIase on its C-terminal side, and a N-terminal domain other than the domain.

It is suggested that, apart from a domain having high homology with parvulin-type PPIase, a N-terminal domain is involved in molecular chaperone activity of the SurA-type PPIase (Behrenns, EMBO J.20, 285-, 2001). In the present invention, PPIase comprising a N-terminal domain of the SurA-type PPIase can be used as PPIase having the molecular chaperone activity. As a N-terminal domain of the SurA-type PPIase, in SEQ ID NO: 7, a region of a N-terminus to around 175^{th} asparagine corresponds to a N-terminal domain. Homology of the N-terminal domain can be determined by using multiple alignment software such as ClustalW.

Both of the FKBP52-type PPIase and CyP40-type PPIase are PPIase found in a eukaryote. The FKBP52-type PPIase is about 52kDa FKBP-type PPIase, and is also called p59 or HSP56. Its amino acid sequence has construction that two regions having high homology with human 12 kDa FKBP-type PPIase are connected in tandem, and a region comprising a calmodulin binding site is connected to its C-terminal sides (Ratajczat, J. Biol. Chem.268, 13187-, 1993). The CyP40-type PPIase is one of cyclophilin-type PPIases having a molecular weight of about 40 kDa and sensitivity to cyclosporine which is an immunological inhibitor. Both of them are characterized in that they have a domain responsible for PPIase activity on its N-terminus, and a domain comprising tetratricopeptide repeat (TPR) binding to HSP90 which is one of heat shock proteins on its C-terminus, and are PPIase involved in formation of a steroid hormone receptor in a eukaryote (Galat, Peptidyl-Prolyl cis/trans isomerase Oxford University Press 1998). The FKBP52-type PPIase and CyP40-type PPIase are not particularly limited, and examples include PPIases derived from a eukaryote such as human, mouse, cow, rabbit and rat. In addition, FKBP52-type PPIase and CyP40-type PPIase used in the present invention are not only PPIase derived from a eukaryote, but also PPIase derived from any organism, as long as it belongs to a group which is recognized to be PPIase having substantially the same function.

As one example of the FKBP52-type PPIase, an amino acid sequence derived from human is shown in SEQ ID NO: 9, and the nucleotide sequence is shown in SEQ ID NO: 10. In addition, as one example of the CyP40-type PPIase, an amino acid sequence derived from human is shown in SEQ ID NO: 11, and the nucleotide sequence is shown in SEQ ID NO: 12.

It has been suggested that each C-terminal domain including TPR is involved in molecular chaperone activity of the FKBP52-type PPIase and CyP40-type PPIase. In the present invention, PPIase comprising a C-terminal domain of FKBP52-type PPIase and CyP40-type PPIase can be used as PPIase having the molecular chaperone activity. In human FKBP52-type PPIase, the aforementioned C-terminal domain is a region of around 264^{th} glutamic acid to a C-terminus in SEQ ID NO: 9 and, among this, a region of 264^{th} glutamic acid to around 400^{th} isoleucine is particularly important. In addition, in human CyP40-type PPIase, a region of around 184^{th} leucine to a C-terminus corresponds to a C-terminal domain.

As PPIase having molecular chaperone activity used in the present invention, PPIase other than the above-exemplified PPIase may be suitably used as long as it is PPIase having equivalent molecular chaperone activity, and examples include pig 18 kDa cyclophilin-type PPIase whose molecular chaperone activity has been recently re-assessed (Ou, Protein Sci. 10, 2346-, 2001).

The expression vector of the present invention comprises (b) a region having at least one restriction enzyme site in which a second coding region encoding a protein can be inserted.

The second coding region is a region having a nucleotide sequence encoding a desired protein which is to be expressed using the expression vector of the present invention.

A second coding region used in the present invention is not particularly limited, and examples include a nucleotide sequence encoding an antibody such as a monoclonal antibody and nucleotide sequences encoding a membrane protein.

The antibody may be an antibody derived from any animal species, and a polypeptide in which a full length antibody, a fragment thereof, or two or more fragments such as Fab, Single chain Fv(scFv) and the like are linked thereto with a linker peptide is included in the antibody. The antibody may be any subclass.

An antibody is a huge molecule having a molecular weight exceeding 100 thousands, is widely used as an analysis reagent or an in vitro diagnostic utilizing function of specifically binding to a specified antigenic substance, and has high industrial utilization value. A part contributing to binding of an antibody molecule and an antigenic substance is called V region (variable region), and is composed of a V region of a heavy chain and a V region of a light chain. As a method of obtaining an antibody to a specified antigen, a method of immunologically sensitizing an experimental animal such as rat and rabbit with an antigenic substance and obtaining an antibody (polyclonal antibody) containing in a serum, and a method of obtaining a monoclonal antibody described below are general.

A monoclonal antibody is an antibody which is produced by an antibody-producing cell of a single clone, and its characteristic is that a primary structure is uniform. A monoclonal antibody can be easily produced by establishment of hybridoma technique by Kohler and Milstein. In this method, first, a predetermined antigenic substance is administered to an experimental animal such as mouse to perform immunological sensitization. Then, a spleen cell which has acquired antibody-producing ability to the antigenic substance is taken out from spleen of an immunologically sensitized animal, and this is fused with an appropriate tumor cell such as myeloma to make a hybridoma. Then, by screening using an appropriate immunological analysis method such as ELISA, a hybridoma producing a desired antibody is selected. Thereafter, by cloning using a limiting dilution method, a hybridoma strain producing a desired monoclonal antibody is established. After the thus established hybridoma is cultured in an appropriate medium, a desired monoclonal antibody is obtained by separating a medium including the metabolite using chromatography etc. However, since these methods utilize an in vivo living body reaction of immunological sensitization of an animal, they inevitably require interposition of an experimental animal. Therefore, an experimental animal must be bred and maintained, troublesome labor is necessary and, at the same time, much cost is necessary. In addition, by this method, a monoclonal antibody to all antigenic substances can not be necessarily produced, and trial and error element is included. Recently, it has become possible to express scFv in which only V regions of a heavy chain and a light chain of an antibody are linked via an appropriate linker, or a Fab part of an antibody, on a superficial layer of Escherichia coli. A method of making an library of an antibody gene by randomly amplifying antibody genes using PCR, making present extracellularly, and screening a member having affinity with a specified antibody from these libraries is being developed (Kumatani et al., Protein-Nucleic Acid-Enzyme 43, 159-, 1998). When the antibody gene obtained by screening is expressed by Escherichia coli or the like, an antibody to a desired antigen can be made without using an experimental animal. However, for example, when an antibody gene is expressed in Escherichia coli at a large amount, almost all was expressed as an insoluble inclusion body, and active type can not be obtained as described above.

To the contrary, when a nucleotide sequence encoding a monoclonal antibody as a second coding region is inserted into the expression vector of the present invention, it becomes possible to easily obtain an active type (soluble type) product of the antibody obtained by screening.

The aforementioned membrane protein is not particularly limited, and examples include a receptor for a physiologically active substance. Since the aforementioned receptor for a physiologically active substance selectively responds to extracellular various substances, and transmits a variety of signals into a cell, this is paid much attention because clarification of the function directly leads to drug generation. These membrane proteins form a structurally well conserved family, and are roughly classified into three of ion channel intrinsic type, tyrosine kinase type and G protein-coupled type. The ion channel intrinsic type is a type such that, when a ligand binds to a receptor, an ion channel present in the receptor itself is opened, Na⁺ and Ca²⁺ are transferred into a cell utilizing an ion gradient between outside and inside a cell. The tyrosine kinase type converts a binding of a ligand into increased in phosphorylation activity to cause a series of cascades, and thereby amplifying a signal. In the G protein-coupled type, a receptor itself has not an ion channel and enzymatic activity, but transmits information by binding of a ligand into a cell via G protein. Many medicaments targeting a membrane receptor protein have been developed, and many of them target a G protein-coupled receptor (GPCR) . Therefore, by specifying an endogenous ligand of GPCR and further clarifying its function and structure, possible rapid development of medicaments can be expected. For crystallization and deuteration for these ligand screening and structural analysis, development of large amount expression technique of GPCR is indispensable, but the previous development of GPCR is impossible in Escherichia coli and yeast. Currently, various analyses are performed using a minor amount of a sample expressed mainly in an animal cultured cell such as CHO, COS-7 and HEK.

To the contrary, when a nucleotide sequence encoding a membrane protein as a second coding region is inserted into the expression vector of the present invention, a recombinant protein can be produced at a low cost and a large amount.

The aforementioned restriction enzyme site is also called multicloning site. A region having the restriction enzyme site is a region in which a gene encoding a desired protein is inserted as a second coding region.

In the expression vector of the present invention (1) the first coding region is operatively linked to a promoter, and the restriction enzyme sites are in the same reading frame as that of the first coding region, and are downstream of the first coding region, or (2) the second coding region in which the restriction enzyme sites are inserted is disposed so that the region is operatively linked to a promoter, and the first coding region is in the same reading flame as that of the second coding region, and is downstream of the second coding region.

The promoter is not particularly limited, and examples include a Plac promoter, a Ptac promoter, a xylA promoter, an AraB promoter, a lambda promoter, a T7 promoter, a gall/gal10 promoter, a nmtl promoter, a polyhedrin promoter, and a mouse metallothionein promoter.

In the expression vector of the present invention, by inserting a second coding region encoding a desired protein into the restriction enzyme sites to obtain an expression vector in which a second coding region is inserted, and expressing this expression vector, (1) when the first coding region is operatively linked to a promoter, and the restriction enzyme sites are in same reading frame as that of the first coding region, and are downstream of the first coding region, a first coding region and, subsequent a second region are translated by the promoter, and on the other hand, (2) when the second coding region in which the restriction enzyme sites are inserted is disposed such that it is operatively linked to a promoter, and the first coding region is in the same reading frame as that of the second coding region, and is downstream of the second coding region, a second coding region and a subsequent a first coding region are translated by the promoter and, in both cases, a desired protein encoded by a second coding region is expressed as a fused protein with a polypeptide having molecular chaperone activity.

The expression vector of the present invention may have a region which is between a region having at least one restriction enzyme site in which the first coding region and the second coding region can be inserted, and which is translated in the same reading frame to become a protease digestion site.

The aforementioned protease digestion site is to be a peptide linker for linking a polypeptide described above and a protein described below, in a fused protein in which a.polypeptide having molecular chaperone activity encoded by first coding region and a protein encoded by a second coding region are bound, which is obtained by expression of an expression vector in which a second coding region is inserted into the expression vector of the present invention. Since the resulting fused protein has a protease digestion site, a protease is acted to easily digest the fused protein, whereby, a polypeptide having molecular chaperone activity encoded by a first coding region and a protein encoded by a second coding region are separated, and a desired protein encoded by a second coding region can be obtained.

The protease is not particularly limited, and examples include thrombin, factor Xa, and precision protease. These proteases are sold by Pharmacia Biotech. Alternatively, a desired protein can be separated utilizing self protein splicing function of intein.

A length of a nucleotide sequence encoding the protease digestion site is not particularly limited, and around 15 to 90 basis is preferable, and it is preferable that many nucleotide sequences which are translated to be a neutral amino acid such as glycine and serine are contained.

The expression vector of the present invention may comprise other known nucleotide sequences. The other known nucleotide sequences are not particularly limited, and examples include a stability leader sequence which imparts stability to an expression product, a signal sequence which imparts secretion of an expression product, and a marking sequence which can impart phenotype selection in a transformed host such as a neomycin resistance gene, a kanamycin resistance gene, a chloramphenicol resistance gene, an ampicillin resistance gene, and a hygromycin resistance gene.

The expression vector of the present invention may be designed into a form that the resulting fused protein binds to a carrier for its immobilization via an appropriate ligand. Thereby, after expression, purification can be simplified. For example, in the case where PPIase is used as a polypeptide having molecular chaperone activity, purification of a fused protein can be simplified by using a carrier harboring FK506, rapamycin or an analogous compound in the case of FKBP-type PPIase, by using a carrier harboring cyclosporin or its analogous compound in the case of cyclophilin-type PPIase, or by using a carrier harboring juglone or its analogous compound in the case of parvulin-type PPIase.

In addition, when the expression vector of the present invention is designed so that it has a tag of about six histidine residues on a N-terminal side of a polypeptide having the molecular chaperone activity, since the resulting fused protein binds to a carrier chelated with a metal such as nickel via a histidine residue, a protein derived from a host and a fused protein can be simply separated using the carrier. Further, by acting a protease on a fused protein bound to the carrier, the aforementioned protease digestion site is digested, and only a desired protein can be simply freed from a carrier.

When eluted with imidazole, a fused protein, as it is, may be freed from a carrier without acting a protease. In addition to the aforementioned histidine tag, a method of purification by affinity chromatography with a glutathione resin using glutathione-s-transferase or a part thereof as a tag, and a method of purification with a maltose resin using a maltose binding protein or a part thereof as a tag may be used. Besides, affinity with an antibody may be used. The aforementioned various tags may be designed on either of a N-terminal side and a C-terminal side of a fused protein, or may be designed on both sides. As these genetic manipulation methods, and affinity purification method, methods known to a person skilled in the art may be used.

By incorporating a gene encoding a desired protein as a second coding region in the expression vector of the present invention to express the gene, a fused protein of a polypeptide having molecular chaperone activity encoded by a first coding region and a protein encoded by a second coding region is obtained. Such the fused protein comprising a polypeptide having the molecular chaperone activity and a protein encoded by a second coding region is also one of the present inventions. Further, when a linker peptide comprising a protease digestion site is introduced between both of them, a desired protein can be easily separated from a fused protein by digesting the resulting fused protein with a protease. For this reason, it is preferable that the fused protein of the present invention comprises a protease digestion site.

The expression vector of the present invention is introduced into a host, and serves for expression of the desired protein. Such the host containing the expression vector of the present invention is one of the present inventions.

The host is not particularly limited, and examples include a prokaryote such as bacterium, yeast, fungus, plant, insect cell, and mammal cell. However, property of a host to be used and that of an expression vector must be adapted. For example, when a fused protein is expressed in a mammal cell system, it is preferable that an expression vector uses a promoter isolated from a genome of a mammal cell such as a mouse metallothionein promoter, or a promoter isolated from virus growing in a mammal cell such as a baculovirus promoter, and a vaccinia virus 7.5K promoter.

As the host, inter alia, a prokaryote such as Escherichia coli is preferable.

A method of introducing the expression vector of the present invention into a host is not particularly limited, and the known various methods can be used, and examples of transfection include a calcium phosphate precipitation method, electroporation, liposome fusion, nuclear injection, and virus or phage infection.

A large amount of a fused protein can be obtained by introducing the expression vector of the present invention into an appropriate host, and culturing the host under appropriate condition to express. Such the process for producing a fused protein of the present invention is also one of the present inventions.

In the process for producing a fused protein of the present invention, it is preferable that a host containing the expression vector of the present invention is cultured under condition of expression of the expression vector to express the fused protein in a cytoplasm, a host containing an expression vector obtained by providing a region which is transcribed and translated to be a signal sequence at a 5′-terminus of a first coding region of the expression vector of the present invention or 5′-terminus of the second coding region is cultured under condition of expression of the expression vector to express the fused protein in a periplasm or a medium, or the expression vector of the present invention is made to express the fused protein in a cell-free translation system.

When a Gram-negative bacterium is used as a host, expression of a fused protein may be expression in a cytoplasm, or in a periplasm or a medium. By providing a signal sequence which is transcribed and translated at a 5′-terminus of a first coding region or 5′-terminus of a second coding region in the expression vector of the present invention, the fused protein can be secreted and expressed in a periplasm or a medium. When the fused protein is expressed in a periplasm, expression property can be improved by using a polypeptide which is originally present in a membrane in a cell as a polypeptide encoded by a first coding region. Examples of the polypeptide which is originally present in a membrane in a cell include FkpA-type PPIase which is FKBP-type PPIase, and SurA-type PPIase which is parvulin-type PPIase. These PPIases are a protein which is originally present in a periplasm of a Gram-negative bacterium, and is involved in folding of a protein.

When a protein encoded by the second coding region is a membrane protein or an antibody, since these proteins are a protein which is originally expressed extracellularly, expression property is improved by expression by fusion with the FkpA-type PPIase or SurA-type PPIase.

When the expression vector of the present invention is made to express the fused protein in a cell-free translation system, the fused protein of the present invention is expressed as a soluble protein in a cell-free translation system using bacteria or eukaryote extract (Spirin, A. S., 1991, Science 11, 2656-2664:Falcone, D. et.al., 1991, Mol. Cell. Biol. 11, 2656-2664) without using a host cell.

In the process for producing the fused protein of the present invention, it is preferable that after a fused protein is adsorbed onto a carrier harboring macrolide, cyclosporin, juglone or its analogous compound which inhibits PPIase, the carrier is recovered.

Binding property between PPIase having the molecular chaperone activity and the aforementioned inhibitor is strong, and an expressed fused protein can be purified utilizing this affinity. For example, when a bead harboring macrolide such as FK506 on an agarose gel carrier is used, a fused protein with FKBP-type PPIase can be specifically bound. Similarly, in the case of a fused protein with cyclophilin-type PPIase, purification can be simplified by using a carrier harboring cyclosporin and, in the case of a fused protein with parvulin-type PPIase, purification can be simplified by using a carrier harboring juglone.

By digesting a fused protein obtained by the process for producing a fused protein of the present invention with a protease which digests a protease digestion site, a desired protein can be obtained. Such the process for producing a protein encoded by the second coding region is also one of the present inventions.

According to the present invention, by linking a desired protein and a polypeptide having molecular chaperone activity with a peptide linker to express as a fused protein, a hardly expressible protein which is originally expressed as an abnormal type can be expressed as a natural type soluble form at a large amount, and its productivity can be improved considerably. In addition, when a desired protein is an antibody, according to the present invention, since it becomes possible to simply produce a recombinant-type antibody having function without using an experimental animal, it becomes possible to produce a highly functional antibody at a large amount by fusion with other protein or peptide.

### BEST MODE FOR CARRYING OUT THE INVENTION

The following Examples further illustrate the present invention in detail, but the present invention is not limited to these Examples.

(EXAMPLE 1) Construction of expression vector for fusion with the short type FKBP-type PPIase (TcFKBP18), which is derived from hyperthermophilic archaebacterium Thermococcus sp. KS-1

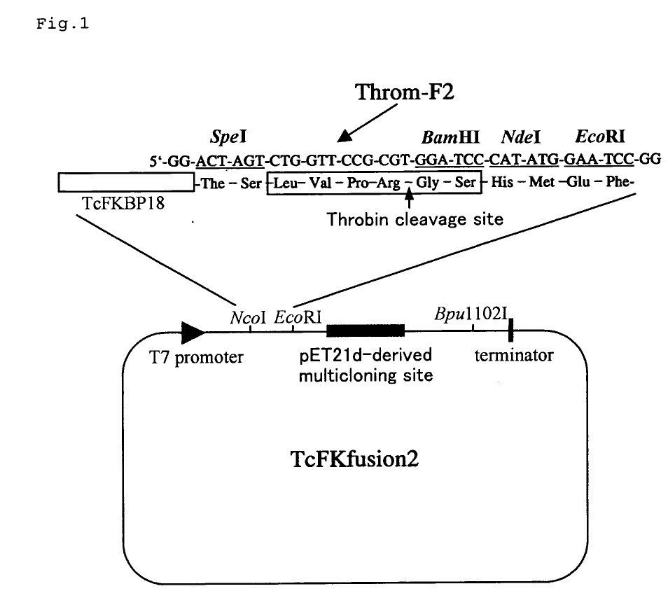

Using an expression plasmid pEFEl-3 (Iida, Gene 222, 249-, 1998) of TcFKBP18 (Ideno, Biochem. J. 357, 465-,2001) having molecular chaperone activity as a template, the TcFKBP18 gene fragment was amplified by a PCR method. By using TcFu-F1 and TcFu-R2 shown in Table 1 as a primer set for PCR, a restriction enzyme site was provided on both sides of the amplification product. On the other hand, as a nucleotide sequence encoding a linker for cutting TcFKBP18 fused protein into TcFKBP18 and a desired protein with a protease, Throm-F2 and its complementary chain were designed. Throm-F2 has a SpeI site on its 5′ side and an EcoRI site on its 3′ side, respectively (FIG. 1). Since Throm-F2 has a BamHI site and a NdeI site downstream of a DNA sequence of a thrombin cleavage site, a fused protein with TcFKBP18 can be obtained by introducing a gene fragment of a desired protein utilizing these restriction enzyme sites (FIG. 1).

A gene fragment of the TcFKBP18 and a DNA fragment encoding a thrombin cleavage site were treated with restriction enzymes respectively, and ligated to a pET21d plasmid DNA which had been treated with NcoI/EcoRI in advance (manufactured by Novagen), in an order of TcFKBP18 gene-Thermo-F2. The resulting plasmid for expressing TcFKBP18 fused protein was designated TcFKfusion2.

TABLE 1

| | | Restriction |
|---|---|---|
| Abbrev. | Sequence | site |
| Tcfu-F1 | 5′-GGCCATGGGAAAAGTTGAAGCTGGTGAT-3′ | Nco I |
| Tcfu-R2 | 5′-CCACTAGTAGCTTCTGAGTCCTCTTC-3′ | Spe I |
| TF-F1 | 5′-GGCCATGGGCCAAGTTTCAGTTGAAACC-3′ | Nco I |
| TF-R1 | 5′-CCACTAGTCGCCTGCTGGTTCATCAGCT-3′ | Spe I |
| FK52-F1 | 5′-GGCCATGGGCACAGCCGAGGAGATGAA-3′ | Nco I |
| FK52-R1 | 5′-CCACTAGTTGCTTCTGTCTCCACCTGA-3′ | Spe I |
| CP40-F1 | 5′-GGCCATGGGCTCGCACCCGTCCCC-3′ | Nco I |
| CP40-R1 | 5′-CCACTAGTAGCAAACATTTTTGCATATACTTG-3′ | Spe I |
| FKPA-F1 | 5′-GGCCATGGGCAAATCACTGTTTAAAGTAACGC-3′ | Nco I |
| FKPA-R1 | 5′-CCACTAGTTTTTTTAGCAGAGTCTGCGGC-3′ | Spe I |
| SUR-F1 | 5′-GGCCATGGGCAAGAACTGGAAAACGCTG-3′ | Nco I |
| SUR-R1 | 5′-CCACTAGTGTTGCTCAGGATTTTAACGTA-3′ | Spe I |
| SCF-F3 | 5′-ATCATATGAAATACCTATTGCCTACG-3′ | Nde I |
| SCF-R3 | 5′-ATGCGGCCGCCATATTACTCCAGCTTGGTCCCTC-3′ | Not I |

Underline: each restriction enzyme site

(EXAMPLE 2) Construction of expression vector for fusion with Escherichia coli trigger factor-type PPIase(TF)

In order to construct an expression vector for fusion with Escherichia coli trigger factor-type PPIase (TF), a TF gene was amplified by PCR from Escherichia coli K12 strain removing a termination codon. By using TF-F1 and TF-R1 shown in Table 1 as a primer set for PCR, restriction enzyme sites were provided on both sides of the amplification product. After insertion of the PCR product into a pT7 blue T vector, it was confirmed that a sequence is not different from registered information. On the other hand, TcFKfusion2 prepared in Example 1 was treated with NcoI/SpeI, and then, a vector in which a TcFKBP18 gene had been removed was purified by agarose gel electrophoresis. A pT7 blue T vector comprising a TF gene was treated with NcoI/SpeI, and an excised TF gene was recovered. The resulting TF gene and the aforementioned vector were ligated to recover a vector comprising a full length TF gene. Thereby, a TF fused protein expression system was constructed in which a TcFKBP18 gene in TcFKfusion2 in Example 1 was replaced with TF gene. The resulting TF fused protein expressing plasmid was designated as TFf2.

(EXAMPLE 3) Construction of expression vector for fusion with human FKBP 52-type PPIase

In order to construct an expression vector for fusion with human FKBP52-type PPIase(hFKBP52), a FKBP52 gene removing a termination codon was amplified by PCR from a human cDNA library. By using FK52-F1 and FK52-R1 shown in Table 1 as a primer set for PCR, restriction enzyme sites were provided on both sides of the amplification product. After insertion of the PCR product into a pT7 blue T vector, it was confirmed that a sequence is not different from registered information. On the other hand, the vector in which a TcFKBP18 gene was removed by treating TcFKfusion2 prepared in Example 1 with NcoI/SpeI, was purified by agarose gel electrophoresis. The pT7 blue T vector comprising a hFKBPS2 gene was treated with NcoI/SpeI, and a fragment of an excised hFKBP52 gene was recovered. The resulting hFKBP52 gene fragment and the aforementioned vector were ligated to recover a vector comprising a full length of hFKBP52 gene. Thereby, an expression system for a fused protein with hFKBP52 was constructed in which a TcFKBP18 gene in TcFKfusion2 of Example 1 was replaced with a hFKBP52 gene. The resulting hFKBP52 fused protein expressing plasmid was designated as FK52f2.

(EXAMPLE 4) Construction of expression vector for fusion with human CyP40-type PPIase

In order to construct an expression vector for fusion with human CyP40-type PPIase (CyP40), a hCyP40 gene removing a termination codon was amplified by PCR from a human cDNA library. By using CP40-F1 and CP40-R1 shown in Table 1 as a primer set for PCR, restriction enzyme sites were provided on both sides of the amplification product. After insertion of the PCR product into a pT7 blue T vector, it was confirmed that a sequence is not different from registered information. On the other hand, TcFKfusion2 prepared in Example 1 was treated with NcoI/SpeI, and the vector from which a TcFKBP18 gene had been removed, was purified by agarose gel electrophoresis. The pT7 blue T vector comprising a hCyP40 gene was treated with NcoI/SpeI, and an excised hCyP40 gene was recovered. The resulting hCyP40 gene and the aforementioned vector were ligated, and a vector comprising a full length hCyP40 gene was recovered. Thereby, a system for expressing a fused protein with hCyP40 was constructed in which the TcFKBP18 gene in TcFKfusion2 of Example 1 was replaced with a hCyP40 gene. The resulting hCyP40 fused protein expressing plasmid was designated as CP40f2.

(EXAMPLE 5) Construction of expression vector for fusion with Escherichia coli FkpA-type PPIase

In order to construct an expression vector for fusion with Escherichia coli FkpA-type PPIase (FkpA), a FkpA gene removing a termination codon was amplified by PCR from Escherichia coli CTF073 strain. By using FKPA-F1 and FKPA-R1 shown in Table 1 as a primer set for PCR, restriction enzyme sites were provided on both sides of the amplification product. After insertion of the PCR product into a pT7 blue T vector, it was confirmed that a sequence is not different from registered information. On the other hand, TcFKfusion2 prepared in Example 1 was treated with NcoI/SpeI, and the vector from which a TcFKBP18 gene had been removed, was purified by agarose gel electrophoresis. The pT7 blue T vector comprising a FkpA gene was treated with NcoI/SpeI, and the excised FkpA gene was recovered. The resulting FkpA gene and the aforementioned vector were ligated, and a vector comprising a full length FkpA gene was recovered. Thereby, a system for expressing a fused protein with FkpA was constructed in which the TcFKBP18 gene in TcFKfusion2 of Example 1 was replaced with a FkpA gene. The resulting FkpA fused protein expressing plasmid was designated as FkpaAf2.

(EXAMPLE 6) Construction of expression vector for fusion with Escherichia coli SurA-type PPIase

In order to construct an expression vector for fusion with Escherichia coli SurA-type PPIase (SurA), a SurA gene removing a termination codon was amplified by PCR from Escherichia coli K12 strain. By using SUR-F1 and SUR-R1 shown in Table 1 as a primer set for PCR, restriction enzyme sites were provided on both sides of the amplification product. After insertion of the PCR product into a pT7 blue T vector, it was confirmed that a sequence is not different from registered information. On the other hand, TcFKfusion2 prepared in Example 1 was treated with NsoI/SpeI, and the vector from which the TcFKBP18 gene had been removed, was purified by agarose gel electrophoresis. The pT7 blue T vector comprising a SurA gene was treated with NcoI/SpeI, and the excised SurA gene was recovered. The resulting SurA gene and the aforementioned vector were ligated, and a vector comprising a full length SurA gene was recovered. Thereby, a system for expressing a fused protein with SurA was constructed in which a TcFKBP18 gene in TcFKfusion2 of Example 1 was replaced with a SurA gene. The resulting SurA fused protein expressing plasmid was designated as SurAf2.

(EXAMPLE 7) Expression of TcFKBP18 using TcFKfusion2 E. coli BL21 (DE3) strain was transformed with TcFKfusion2 prepared in Example 1. 700 mL of a 2×YT medium (Yeast Extract 16 g/L, BACTO TRYPTON 20 g/L, NaCl 5 g/L, ampicillin 100 μg/mL, pH 7.5) was placed into a 2L Erlenmeyer flask, and this was inoculated with 2 to 3 platinum loops of recombinant Escherichia coli. After rotation culturing (110 rpm) at 35° C. for 24 hours, cells were recovered by centrifugation (10000 rpm×10 min). The resulting cells were suspended in 20 mL of a 25 mM HEPES buffer (pH 6.8) containing 1 mM EDTA, and freezing-stored at −20° C.

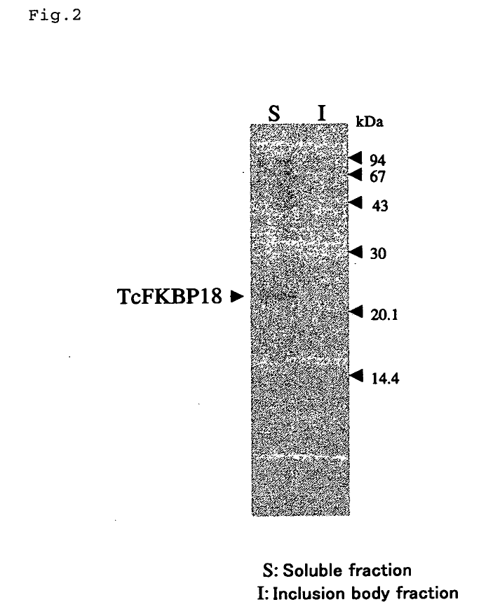

The resulting cells were disrupted by sonication, and centrifuged to separate into the supernatant (soluble fraction) and the precipitate part (precipitate fraction). In order to further purify to an inclusion body fraction, the precipitate fraction was suspended in a 25 mM HEPES/1 mM EDTA buffer (pH 6.8) containing 4% Triton X-100, and thereafter the materials were reacted for 30 minutes to solubilize membrane components, and precipitated inclusion body components were recovered by centrifugation. This procedure was repeated two times, and the resulting precipitated part was regarded as an inclusion body fraction. 10 μg of a soluble fraction and a volume of an inclusion body fraction corresponding it were subjected to 16% SDS-PAGE. As a result, a band corresponding to TcFKBP18 was seen only in a soluble fraction. A band was detected at a position corresponding to a higher apparent molecular weight than a position at which TcFKBP18 is originally detected (FIG. 2). The reason can be considered as follows: Since a termination codon is not present at a 3′ terminus of a structural gene of TcFKBP18, and a multicloning site is present there, the translation product is connected to a C-terminus of TcFKBP18.

(EXAMPLE 8) Expression of fused protein composed of TcFKBP18 and mouse anti-hen egg white lysozyme (HEL) Fab antibody fragment (D1.3)

An expression plasmid pEHELFab-1 (Ideno, Appl. Env. Microbiol.68, 464-, 2002) for a mouse anti-HEL Fab antibody fragment was treated with NdeI/Bpul102I, and an anti-HEL Fab antibody fragment gene fragment was purified by an electrophoresis method using an agarose gel. This DNA fragment was ligated to TcFKfusion2 which had been treated with NdeI/Bpul102I in advance. When the plasmid obtained as this result was expressed, a heavy chain part of the Fab was expressed as a fused protein with TcFKBP18, and it was found that a light chain part is expressed alone not as a fused protein. The resulting plasmid was introduced into Escherichia coli in the same method as that of Example 7, and a transformant was obtained. This bacterium was cultured and recovered in the same manner as that of Example 7, and freezing-stored at −20° C.

The resulting cells were disrupted by sonication, and centrifuged to separate into the supernatant (soluble fraction) and the precipitate part (precipitate fraction), which were subjected to SDS-PAGE in the same manner as that of Example 7. SDS-PAGE gel was stained with Coomassie Brilliant Blue (CBB) and the expressed Fab was specifically detected by a Western blotting method using a rabbit anti-D1.3 antibody as a primary antibody.

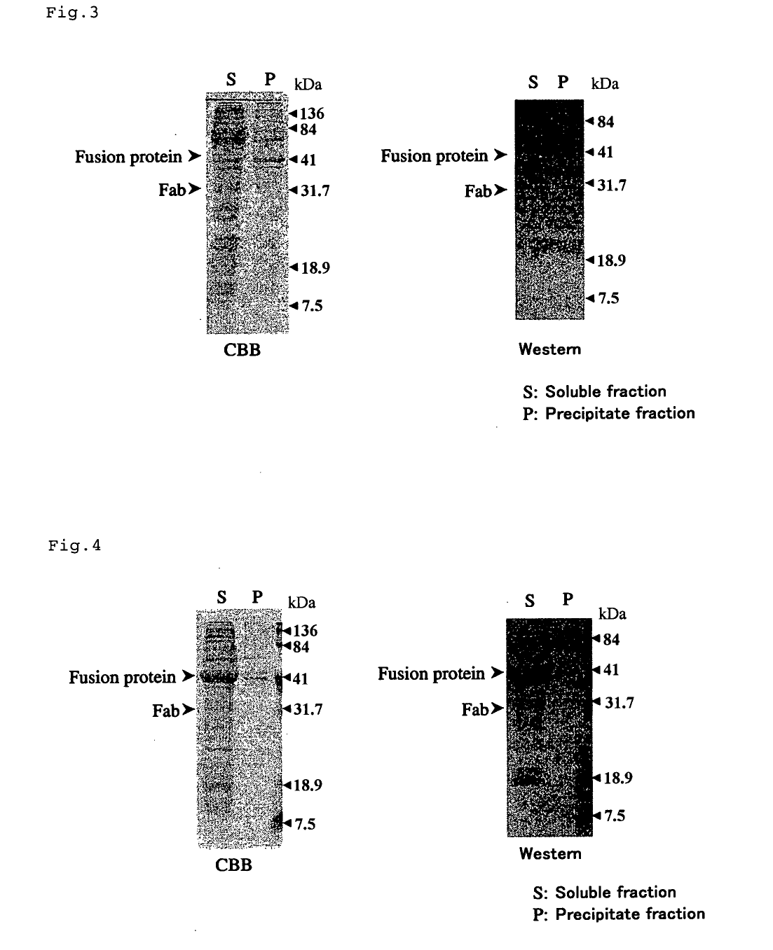

A band corresponding to a fused protein of Fab and TcFKBP18 was not seen in a soluble fraction and a precipitate fraction of Escherichia coli which is a host bacterium, in detection either of CBB staining or Western blotting (FIG. 3). On the other hand, when expressed as a fused protein with TcFKBP18, it was shown that a heavy chain part of Fab is expressed as a major band in a soluble fraction as a form fused with TcFKBP18, in CBB staining, and it was revealed that Fab is assuredly expressed at a large amount also in Western blotting (FIG. 4). In CBB staining, a band density of a fused protein expressed in a soluble fraction was measured with a densitometer and, as a result, the density was about 10% of all soluble proteins derived from Escherichia coli. To the contrary, a band corresponding to a light chain part of Fab was not detected. From the result of Western blotting, it was considered that a light chain of Fab is degraded with a host protease (FIG. 4).

(COMPARATIVE EXAMPLE 1) Expression of mouse anti-HEL Fab antibody fragment alone

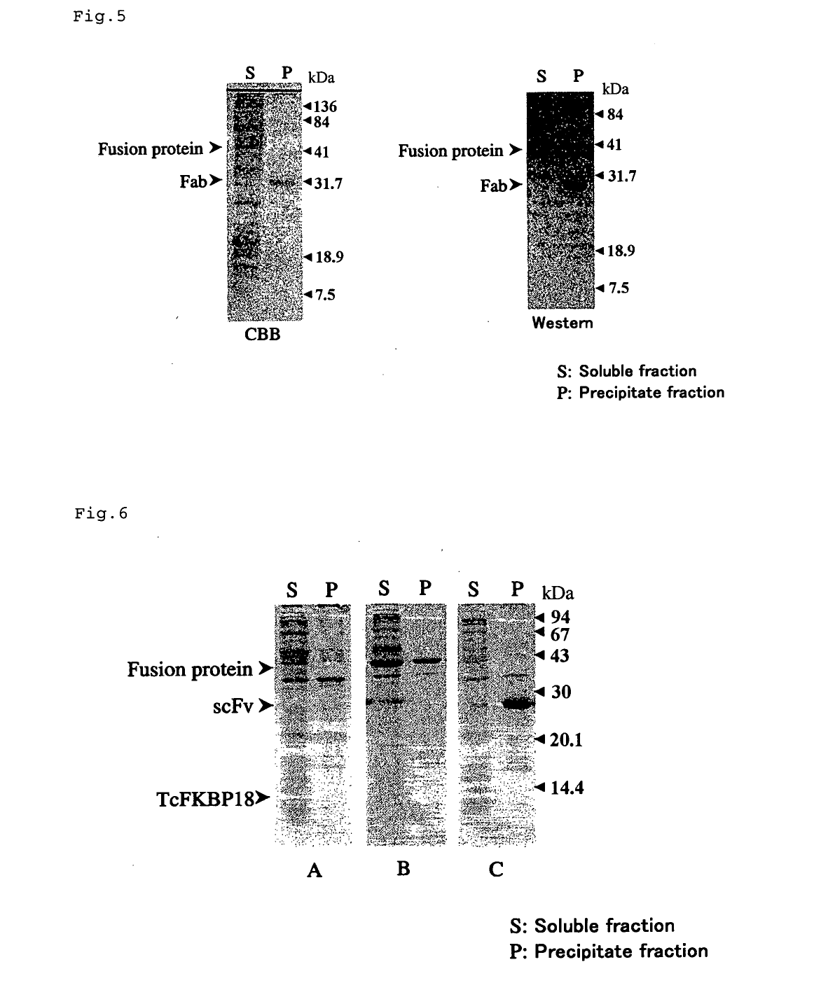

An expression plasmid pEHELFab-1 for mouse anti-HEL Fab antibody fragment was introduced into Escherichia coli in the same manner as that of Example 7, and evaluated with SDS-PAGE. As a result of CBB staining and Western blotting, it was confirmed that a Fab gene alone is not expressed in a soluble fraction, and all is expressed in a precipitate fraction (FIG. 5).

(EXAMPLE 9) Expression of fused protein of mouse anti-HEL scFv and TcFKBP18

By PCR using SCF-F3 and SCF-R3 shown in Table 1 as a primer set and using an expression plasmid pAALSC for mouse anti-HEL scFv fragment (Iba et al. 1997, Gene 194, 35-) as a template, a gene of a mouse anti-HEL scFv fragment was amplified. This gene was ligated to a pT7 blue vector by TA cloning, treated with NdeI/NotI, and then ligated again to TcFKfusion2 which had been treated with the same restriction enzymes in advance, to construct a system for expressing a fused protein of TcFKBP18 and scFv. The resulting plasmid was introduced into Escherichia coli in the same manner as that of Example 7, and a transformant was obtained. This bacterium was cultured and recovered in the same manner as that of Example 7, and freezing-stored at −20° C. The resulting cell solution was subjected to SDS-PAGE in the same manner as that of Example 7, and stained with CBB.

A band corresponding to a fused protein of mouse anti-HEL scFv and TcFKBP18 was not detected in a soluble fraction and a precipitate fraction of Escherichia coli which is a host bacterium, in CBB staining (FIG. 6A). On the other hand, when expressed as a fused protein with TcFKBP18, it was shown that mouse anti-HEL scFv was expressed as a major band in a soluble fraction as a form fused with TcFKBP18, at a large amount (FIG. 6B). In CBB staining, a band density of a fused protein expressed in a soluble fraction was measured with a densitometer and, as a result, the density was about 14% of all soluble proteins derived from Escherichia coli.

(COMPARATIVE EXAMPLE 2) Expression of mouse anti-HEL scFv alone

The pT7 blue vector comprising a mouse anti-HEL scFv gene obtained in Example 9 was treated with NdeI/NotI, and then ligated again to pET21a (manufactured by Novagen) which had been treated with the same restriction enzymes in advance, whereby, a system for expressing mouse anti-HEL scFv was constructed. The resulting expression plasmid was introduced into Escherichia coli according to the same manner as that of Example 9, and a transformant was obtained. This bacterium was cultured and recovered in the same manner as that of Example 7, and freezing-stored at −20° C. The resulting cell solution was subjected to SDS-PAGE in the same manner as that of Example 7, and stained with CBB. As a result, it was confirmed that mouse anti-HEL scFv is hardly expressed in a soluble fraction, and almost all is expressed in an insoluble fraction (FIG. 6C).

(EXAMPLE 10) Expression of fused protein of mouse anti-HEL scFv and TF

A NdeI/NotI-treated DNA fragment of the pT7 blue vector comprising mouse anti-HEL scFv fragment prepared in Example 9 was ligated to the TFf2 of Example 2 which had been treated with the same restriction enzymes in advance, whereby, a system for expressing a fused protein of TF and scFv was constructed. The resulting plasmid was introduced into Escherichia coli in the same manner as that of Example 7, and a transformant was obtained. This bacterium was cultured and recovered in the same manner as that of Example 7, and freezing-stored at −20° C. The resulting cell solution was subjected to SDS-PAGE in the same manner as that of Example 7, and stained with CBB. When expressed as scFv alone, scFv is expressed in an insoluble fraction an shown in Comparative Example 2, while when expressed as a fused protein with TF, it was found that scFv is expressed in a soluble fraction at a large amount. In CBB staining, a band density of a fused protein expressed in a soluble fraction was measured with a densitometer and, as a result, the density was about 7% of all soluble proteins derived from Escherichia coli.

(EXAMPLE 11) Expression of fused protein of mouse anti-HEL scFv and hFKBP52

The vector for expressing a fused protein of TcFKBP18 and scFv prepared in Example 9 was treated with SpeI/NotI, and a DNA fragment comprising a scFv fragment was prepared. By ligating the DNA fragment to FK52f2 of Example 3 which had been treated with the same restriction enzymes in advance, a system for expressing a fused protein of hFKBP52 and scFv was constructed. The resulting plasmid was introduced into Escherichia coli in the same manner as that of Example 7, and a transformant was obtained. This bacterium was cultured and recovered in the same manner as that of Example 7, and freezing-stored at −20° C. The resulting cell solution was subjected to SDS-PAGE in the same manner as that.of Example 7, and stained with CBB. It was found that when expressed as a fused protein with hFKBP52, this is expressed in a soluble fraction at a large amount. In CBB staining, a band density of a fused protein expressed in a soluble fraction was measured with a densitometer and, as a result, the density was about 9% of all soluble proteins derived from Escherichia coli.

(EXAMPLE 12) Expression of fused protein of mouse anti-HEL scFv and hCyP40

According to the same manner as that of Example 11 except that the CP40f2 prepared in Example 4 was used in place of the FK52f2 of Example 11, a fused protein of hCyP40 and scFv was expressed. In CBB staining, a band density of a fused protein expressed in a soluble fraction was measured with a densitometer and, as a result, the density was about 11% of all soluble proteins derived from Escherichia coli.

(Eaxample 13) Expression of fused protein of human serotonin receptor and FkpA

In order to construct a system for expressing a fused protein of a human serotonin receptor (HTla receptor) which is one of seven times transmembrane-type proteins, and Escherichia coli FkpA, a HTla receptor gene was cloned from a human cDNA library. That is, primers for PCR were designed based on nucleotide sequence information registered as NCBI code: HSSERR51, and a HTla receptor gene was amplified by PCR using a human cDNA library as a template.

An amino acid sequence of HTla is shown in SEQ ID NO: 13, and a nucleotide sequence is shown in SEQ ID NO: 14. An NdeI restriction enzyme site was provided on a 5′ side of a primer, and a NotI restriction enzyme site was provided on a 3′ side, respectively. After insertion of the PCR product into a pT7 blue T vector, it was confirmed that a sequence is not different from registered information. A DNA fragment comprising a HTla gene was cut out and purified by NdeI/NotI treatment, ligated to the FkpAf2 of Example 5 which had been treated with NdeI/NotI in advance, and a vector comprising a HTla gene was recovered. The resulting vector for expressing a fused protein of FkpA and HTla was introduced into Escherichia coli in the same manner as that of Example 7, and a transformant was obtained. This bacterium was cultured and recovered in the same manner as that of Example 7, and freezing-stored at −20° C. The resulting cells were disrupted by sonication, and centrifuged at 3000 rpm to fractionate into the supernatant (soluble fraction) and the precipitate part (precipitate fraction). This was subjected to SDS-PAGE in the same manner as that of Example 7, and expressed HTla was detected by staining with Coomassie Brilliant blue (CBB) and a Western blotting method specifically using an anti-serotonin receptor antibody. As a result, in CBB staining, it was shown that HTla is expressed in a soluble fraction as a form fused with FkpA, and it was confirmed that HTla is assuredly expressed also in Western blotting. In CBB staining, a band density of a fused protein expressed in a soluble fraction was measured with a densitometer and, as a result, the density was about 2% of all soluble proteins derived from Escherichia coli.

(EXAMPLE 14) Expression of fused protein of human serotonin receptor and SurA

According to the same manner as that of Example 13 except that the SurAf2 prepared in Example 6 was used in place of the FkpAf2 of Example 13, a fused protein of SurA and HT1a was expressed.

In the same manner as that of Example 7, this was subjected to SDS-PAGE, the expressed HT1a was detected by staining with Coomassie Brilliant Blue (CBB) and a Western blotting method specifically using an anti-serotonin receptor antibody, and it was shown that, in CBB staining, HT1a is expressed in a soluble fraction as a form fused with SurA, and it was confirmed that HT1a is assuredly expressed also in Western blotting. In CBB staining, a band density of a fused protein expressed in a soluble fraction was measured with a densitometer and, as a result, the density was about 2% of all soluble proteins derived from Escherichia coli.

(EXAMPLE 15) Purification of fused protein of mouse anti-HEL scFv and TcFKBP18

By repeating column-purification of the soluble fraction obtained in Example 9 in an order of the following (a) and (b) anion exchange chromatography and gel filtration, a fused protein of mouse anti-HEL scFv and TcFKBP18 was purified to almost single. An amount of the fused protein obtained as the result of purification was about 50 mg per 1 L of a medium.
(a) DEAE Toyopearl column (16 mm×60 cm; manufactured by Tosoh Corporation)

Solution A: 25 mM HEPES-KOH buffer (pH 6.8)

Solution B: 25 mM HEPES-KOH buffer (pH 6.8) containing 0.5 M NaCl

(0 to 300 min: Solution B 0 to 100% linear gradient, 300 to 420 min: Solution B 100%)

Flow rate: 1 mL/min
(b) HiLoad 26/60 Superdex 200 pg column (26 mm×60 cm; manufactured by Amersham Pharmacia)

Eluent: 100 mM sodium phosphate buffer (pH 7.0; containing 0.15 M NaCl)

Flow rate: 3 mL/min

(EXAMPLE 16) Cleavage of fused protein with thrombin

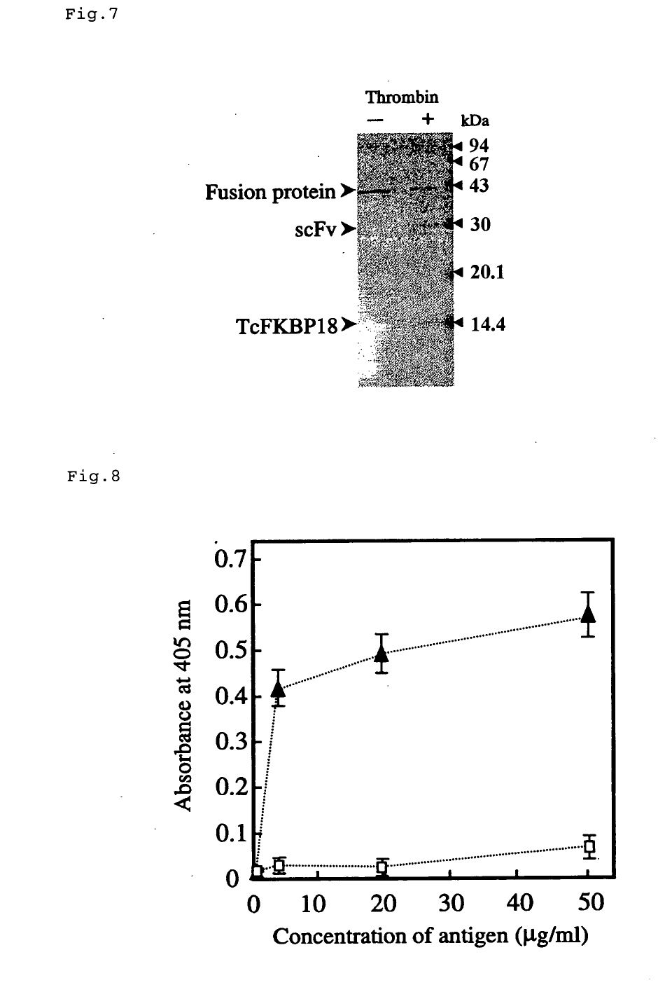

Thrombin was added at 1OU per 1 mg of the fused protein purified in Example 15, and treated at 22° C. for 16 hours, whereby, a thrombin site of the fused protein was cleaved. As a result of SDS-PAGE, it was confirmed that the fused protein was assuredly cleaved into mouse anti-HEL scFv and TcFKBP18 (FIG. 7).

(EXAMPLE 17) Confirmation of function of mouse anti-HEL scFv by ELISA

Function of mouse anti-HEL scFv obtained by expression was assessed by whether it functions as a primary antibody or not in an ELISA method using hen egg white lysozyme as an antigen. That is, 100 μL of a 50 μg/mL hen egg white lysozyme (HEL) solution was added to a 96-well plate, and incubated at 30° C. for 3 hours to immobilize HEL onto the plate. After the plate was washed with a TBS buffer (pH 7.0), this was blocked with a TBS buffer containing Block Ace (manufactured by Dainippon Pharmaceutical Co., Ltd.) (at 4° C., overnight). After washed with TBS, incubation was performed at room temperature for 3 hours using TBS (containing 10% Block Ace) containing 24 μg of mouse anti-HEL scFv obtained in Example 16. After washed with TBS, incubation (2 hours, 30° C.) was performed using a TBS buffer comprising an Anti-mouse IgG-HRP conjugated (manufactured by Funakoshi Co., Ltd) as a secondary antibody. After washed with TBS, 100 μL of an ABTS solution (manufactured by Funakoshi Co., Ltd) as a substrate for HRP was added, followed by incubation for 30 minutes and measurement of OD405. The obtained results are shown in FIG. 8. Since absorbance is increased (▴) depending on a concentration of HEL immobilized on a plate, it was confirmed that the resulting scFv is bound with an antigen. On the other hand, when the same concentration of chymotrypsin inhibitor (□) was used in place of HEL, increase in absorbance was not observed. It seems that this demonstrates that the antibody obtained by expression is specifically bound with the antigen.

### INDUSTRIAL APPLICABILITY

Since the present invention comprises the aforementioned essential features, it enables to prevent formation of an inclusion body which has previously been a problem in a protein expression system using bacteria, yeast and insect cell, and express a native-type protein in a soluble fraction at a large amount. Whereby, labor of re-folding an inclusion body into a native-type protein in vitro as previous becomes unnecessary.

1-32. (canceled)

33. An expression vector, which comprises:
(a) a first coding region encoding PPIase having molecular chaperone activity, and
(b) a region having at least one restriction enzyme site in which a second coding region encoding a desired protein can be inserted.

34. The expression vector according to claim 33,
wherein the first region is operatively linked to a promoter, and the restriction enzyme site is in the same reading frame as the first coding region, and is downstream of the first coding region.

35. The expression vector according to claim 33,
which has a region being between a first coding region and a region having at least one restriction enzyme site in which a second coding region can be inserted, and is translated in the same reading frame to be a protease digestion site.

36. An expression vector,
wherein a second coding region encoding a desired protein is inserted into the expression vector according to claim 33.

37. The expression vector according to claim 33,
wherein the PPIase having molecular chaperone activity is FKBP-type PPIase.

38. The expression vector according to claim 33,
wherein the PPIase having molecular chaperone activity is cyclophilin-type PPIase.

39. The expression vector according to claim 33,
wherein the PPIase having molecular chaperone activity is parvulin-type PPIase.

40. The expression vector according to claim 37,
wherein the FKBP-type PPIase is archaebacterial FKBP-type PPIase.

41. The expression vector according to claim 40,
wherein the archaebacterial FKBP-type PPIase is short type FKBP-type PPIase.

42. The expression vector according to claim 33,
wherein the PPIase having molecular chaperone activity comprises an IF domain and/or a C-terminal domain of archaebacterial FKBP-type PPIase.

43. The expression vector according to claim 37,
wherein the FKBP-type PPIase is trigger factor-type PPIase.

44. The expression vector according to claim 33,
wherein the PPIase having molecular chaperone activity comprises a N-terminal domain and/or a C-terminal domain of trigger factor-type PPIase.

45. The expression vector according to claim 37,
wherein the FKBP-type PPIase is FkpA-type PPIase.

46. The expression vector according to claim 33,
wherein the PPIase having molecular chaperone activity comprises a N-terminal domain of FkpA-type PPIase.

47. The expression vector according to claim 37,
wherein the FKBP-type PPIase is FKBP52-type PPIase.

48. The expression vector according to claim 33,
wherein the PPIase having molecular chaperone activity comprises a C-terminal domain of FKBP52-type PPIase.

49. The expression vector according to claim 38,
wherein the cyclophilin-type PPIase is CyP40-type PPIase.

50. The expression vector according to claim 33,
wherein the PPIase having molecular chaperone activity comprises a C-terminal domain of CyP40-type PPIase.

51. The expression vector according to claim 39,
wherein the parvulin-type PPIase is SurA-type PPIase.

52. The expression vector according to claim 33,
wherein the PPIase having molecular chaperone activity comprises a N-terminal domain of SurA-type PPIase.

53. The expression vector according to claim 36,
wherein the second coding region has a nucleotide sequence encoding a monoclonal antibody.

54. The expression vector according to claim 36,
wherein the second coding region has a nucleotide sequence encoding a membrane protein.

55. A host,
which contains the expression vector according to claim 33.

56. The host according to claim 55, which is Escherichia coli.

57. A fused protein,
which comprises PPIase having molecular chaperone activity and a desired protein.

58. The fused protein according to claim 57,
which comprises a protease digestion site between PPIase having molecular chaperone activity and a desired protein.

59. A process for producing a fused protein comprising PPIase having molecular chaperone activity and a desired protein,
which comprises making the expression vector according to claim 36, express the fused protein.

60. The process for producing a fused protein according to claim 59,
which comprises culturing the host containing the expression vector under condition of expression of the expression vector, and making express the fused protein in a cytoplasm.

61. The process for producing a fused protein according to claim 59,
which comprises providing a region being transcribed and translated to be a signal sequence at a 5′ terminus of a first coding region or a 5′ terminus of a second coding region of the expression vector, and culturing a host containing the expression vector under condition of expression of the expression vector to express the fused protein in a periplasm or a medium.

62. The process for producing a fused protein according to claim 59,
which comprises making the expression vector express the fused protein in a cell-free translation system.

63. The process for producing a fused protein according to claim 59,
wherein the fused protein is adsorbed on a carrier harboring macrolide, cyclosporin, juglone or its analogous compound inhibiting PPIase activity, and then the carrier is recovered and the fused protein is recovered from the carrier.

64. A process for producing a desired protein,
which comprises digesting the fused protein comprising a protease digestion site obtained by the process according to claim 59, 60, 61, 62 or 63, with a protease digesting a protease digestion site.
